(19) United States
(12) Patent Application Publication
(10) Pub. No.: US 2008/0214508 A1
(43) Pub. Date: Sep. 4, 2008

(54) Methods of Administering Water-Soluble Prodrugs of Propofol

(75) Inventors: Barbara S. Slusher, Kingsville, MD (US); Krystyna Wozniak, Bel Air, MD (US)

Correspondence Address: BANNER & WITCOFF, LTD., 1100 13th STREET, N.W., SUITE 1200, WASHINGTON, DC, 20005-4051, US

(73) Assignee: MGI GP, INC., Wilmington, DE (US)

(21) Appl. No.: 11/661,990

(22) PCT Filed: Sep. 14, 2005
(86) PCT No.: PCT/US2005/032701

§ 371 Date: Mar. 4, 2008

Related U.S. Application Data
(60) Provisional application No. 60/610,786, filed on Sep. 17, 2004.

(51) Int. Cl. A61K 31/661; A61P 25/08; A61P 43/00; A61P 25/00; A61P 1/00

(52) U.S. Cl. 514/130

(57) ABSTRACT

A method of administering a prodrug of propofol, preferably O-phosphonooxymethyl propofol disodium salt, comprises the subcutaneous or rectal administration of the prodrug in amounts sufficient to induce or maintain a generalized anesthetized state, a conscious sedated state, or to treat insomnia, anxiety, nausea, vomiting, pruritus, epilepsy, and a range of pain syndromes, including migraine pain, and other medical conditions.

### BACKGROUND OF THE INVENTION

Propofol (2,6-diisopropylphenol) is a low molecular weight phenol derivative that is widely used as a hypnotic or sedative agent for intravenous administration in the induction and maintenance of anesthesia or sedation in humans and animals. Among its useful characteristics as an anesthetic drug are: administration via the intravenous route, rapid onset and offset of anesthesia, rapid clearance, and a side-effect profile that makes it preferable to other injectable anesthetics, such as barbiturates.

The use of injectable anesthetic agents generally, and of propofol specifically, in the induction and maintenance of general anesthesia has gained widespread acceptance in anesthetic care over the last 15 years. Intravenous anesthesia with propofol has been described to have several advantages over preexisting methods, such as more readily tolerated induction, since patients need have no fear of masks, suffocation, or the overpowering smell of volatile anesthetics; rapid and predictable recovery; readily adjustable depth of anesthesia by adjusting the IV dose of propofol; a lower incidence of adverse reactions as compared to inhalation anesthetics; and decreased dysphoria, nausea, and vomiting upon recovery from anesthesia [Padfield NL, Introduction, history and development. In: Padfield NL (Ed.) Ed., Total Intravenous Anesthesia. Butterworth Heinemann, Oxford 2000].

In addition to its sedative and anesthetic effects, propofol has a range of other biological and medical applications. For example, it has been reported to be an anti-emetic [McCollum J S C et al., Anesthesia 43 (1988) 239], an anti-epileptic [Chilvers C R, Laurie P S, Anesthesia 45 (1990) 995], and an anti-pruritic [Borgeat et al., Anesthesiology 76 (1992) 510]. Anti-emetic and anti-pruritic effects are typically observed at subhypnotic doses, i.e. at doses that achieve propofol plasma concentrations lower than those required for sedation or anesthesia. Antiepileptic activity, on the other hand, is observed over a wider range of plasma concentrations [Borgeat et al., Anesthesiology 80 (1994) 642]. Short-term intravenous administration of subanesthetic doses of propofol has also been reported to be remarkably effective in the treatment of intractable migraine and nonmigrainous headache [Krusz J C, et al., Headache, 40 (2000) 224-230]. It has further been speculated that propofol may be useful as an anxiolytic [Kurt et al., Pol. J. Pharmacol. 55 (2003) 973-7], neuroprotectant {Velly et al., Anesthesiology 99 (2003) 368-75], muscle relaxant [O'Shea et al., J. Neurosci. 24 (2004) 2322-7] and, due to its antioxidant properties in biological systems, may further be useful in the treatment of inflammatory conditions, especially inflammatory conditions with a respiratory component, and in the treatment of neuronal damage related to neurodegeneration or trauma. Such conditions are believed to be associated with the generation of reactive oxygen species and therefore amenable to treatment with antioxidants [see, e.g. U.S. Pat. No. 6,254,853 to Hendler et al.]

Propofol typically is formulated for clinical use as a oil-in-water emulsion. The formulation has a limited shelf-life and has been shown to be sensitive to bacterial or fungal contamination, which has led to instances of postsurgical infections [Bennett S N et al., N Engl J Med 333 (1995) 147]. Due to the dense, white color of the formulation, bacterial or fungal contamination cannot be detected by visual inspection of the vial in the first instance.

Not only is propofol poorly water soluble, but it also causes pain at the injection site, which must often be alleviated by using a local anesthetic [Dolin S J, Drugs and pharmacology. In: N. Padfield, Ed., Total Intravenous Anesthesia. Butterworth Heinemann, Oxford 2000]. Due to its formulation in a lipid emulsion, its intravenous administration is also associated with undesirable hypertriglyceridemia in patients, especially in patients receiving prolonged infusions [Fulton B and Sorkin E M, Drugs 50 (1995) 636]. Its formulation as a lipid emulsion further makes it difficult to co-administer other IV drugs. Any physical changes to the formulation, such as a change in lipid droplet size, can lead to changes in the pharmacological properties of the drug and cause side effects, such as lung embolisms.

It has further been reported that the use of propofol in anesthesia induction is associated with a significant incidence of apnea, which appears to be dependent on dose, rate of injection, and premedication [Reves, J G, Glass, P S A, Lubarsky D A, Nonbarbiturate intravenous anesthetics. In: R. D. Miller et al., Eds, Anesthesia. 5^{th} Ed. Churchill Livingstone, Philadelphia, 2000]. Respiratory consequences of administering anesthetic induction doses of propofol, including a reduction in tidal volume and apnea, occur in up to 83% of patients [Bryson et al., Drugs 50 (1995) at 520]. Induction doses of propofol are also known to have a marked hypotensive effect, which is dose- and plasma concentration-dependent [Reves et al., supra]. The hypotension associated with peak plasma levels after rapid bolus injection of propofol sometimes requires the use of controlled infusion pumps or the breaking-up of the induction bolus dose into several smaller incremental doses. Further, the short duration of unconsciousness caused by bolus induction doses renders propofol suitable for only brief medical procedures. For all the above reasons, propofol for induction and/or maintenance of anesthesia must normally be administered under the supervision of an anesthesiologist, and is often considered inappropriate for use by non-anesthesiologists in an ambulatory or day case setting.

In addition to its use in induction and maintenance of anesthesia, propofol has been used successfully as a sedative to accompany either local or regional anesthesia in conscious patients. Its sedative properties have also been exploited in diagnostic procedures that have an unsettling effect on conscious patients, such as colonoscopy or imaging procedures. Propofol has also been used as a sedative in children undergoing diagnostic imaging procedures or radiotherapy. A recent development is that of patient-controlled sedation with propofol. This technique is preferred by patients and is as effective as anesthesiologist-administered sedation.

Compared with the widely used sedative midazolam or other such agents, propofol provided similar or better sedative effects when the quality of sedation and/or the amount of time that patients were at adequate levels of sedation were measured [see Fulton B and Sorkin E M, Drugs 50 (1995) 636]. The faster recovery and similar or less amnesia associated with propofol makes it an attractive alternative to other sedatives, particularly for patients requiring only short sedation. However, because of the potential for hyperlipidemia associated with the current propofol formulation, and the development of tolerance to its sedative effects, the usefulness of propofol for patients requiring longer sedation is less well established.

Due to its very low oral bioavailability, propofol in its commercially available formulations is widely recognized as not suitable for other than parenteral administration, and must generally be injected or infused intravenously. While propofol is administered intravenously in a clinical setting, it has been suggested that it could be delivered for certain indications via other non-oral routes, such as via inhalation using a nebulizer, transmucosally through the epithelia of the upper alimentary tract, or rectally in the form of a suppository [see, e.g. Cozanitis, D. A., et al., Acta Anaesthesiol. Scand. 35 (1991) 575-7; see also U.S. Pat. Nos. 5,496,537 and 5,288,597]. However, the poor bioavailability of propofol when administered by any other than the intravenous route has hampered the development of such treatments. Alternative, safe, and simple methods of administration of propofol which do not require intravenous injections or infusions would be highly useful in a non-clinical setting for the treatment of conditions such as, for example, migraine and other severe headaches, trigeminal facial or dental pain, or arachnoiditis, to achieve mild sedation, anxiolysis, suppression of nausea, or as a sleep aid in individuals in need thereof. International patent application publication WO 02/13810 to Hendler teaches several propofol or di-propofol phosphate esters and carboxylic hemiesters of propofol which are disclosed as water-soluble and useful in the treatment of migraine.

Methods allowing for the delivery of therapeutic doses of propofol by other than the intravenous route would be highly beneficial. To date, however, these medical needs have gone largely unmet. For all the reasons given above, there continues to exist a clear clinical need for stable formulations of safe agents in anesthetic care which are bioavailable by other than the intravenous route, and for the use of such formulations and delivery methods in the treatment of conditions such as, for example, epilepsy, pruritus, migraine and other severe headaches, and nausea and vomiting.

The development of water soluble and stable prodrugs of propofol, which is described in U.S. Pat. No. 6,204,257 to Stella et al., has made it possible to address these heretofore unmet needs, and to explore the pharmaceutical advantages of an aqueous propofol-prodrug as a therapeutic agent. The prodrugs of the present invention differ from propofol in that the 1-hydroxy-group of propofol is replaced with a phosphonooxymethyl ether group:

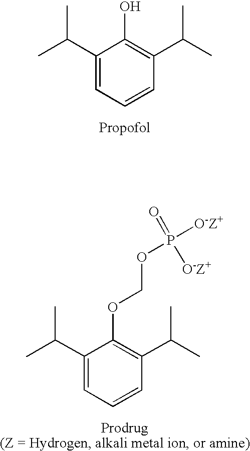

While the present invention is not bound by any theory, the prodrug is believed to undergo hydrolysis by endothelial cell surface alkaline phosphatases to release propofol.

Stella reports that the prodrug has good stability at pH levels suitable for making pharmaceutical formulations, and quickly breaks down in vivo under physiological conditions when administered intravenously. International patent application publication WO 03/086413 to Wingard et al. discloses that plasma propofol derived from intravenous infusions of the prodrug was significantly more potent in suppressing EEG activity and causing a hypnotic effect in human subjects than was plasma propofol derived from infusions of propofol itself. Unexpectedly, the inventors have now found that the prodnig displays pharmacodynamic properties that make it well-suited for medical use when administered not only intravenously, but also by the therapeutically convenient subcutaneous or rectal routes. For example, in contrast to propofol itself, the prodrug can be administered subcutaneously to achieve a condition ranging from mild sedation and reduced responsiveness to external stimuli to deep sedation and loss of consciousness, depending on the subcutaneously administered dose of the prodrug. When dosed by this route, the prodrug causes a rapid onset of the sedated/unconscious state, followed by a plateau effect which is reached within about 10-15 minutes after administration and is, depending on the administered dose, maintained for up to about thirty minutes to two hours or longer.

Yet another finding of the inventors is that the prodrug of the invention displays good bioavailability sufficient to cause therapeutically useful plasma concentrations of propofol when administered subcutaneously or rectally. When a single dose of the prodrug is administered by these routes, peak propofol plasma concentrations are lower than after a substantially similar single intravenous dose; however, therapeutically appreciable propofol plasma concentrations are sustained over a longer time. The prodrugs of the invention thus possess excellent and unexpected properties for therapeutically convenient administration via the subcutaneous and rectal routes, and a favorable pharmacological profile as subcutaneously or rectally bioavailable therapeutics for sedation and anesthetic care, and for the treatment of conditions such as migraine, epilepsy, pruritus, anxiety, insomnia, nausea, and other medical conditions.

### BRIEF SUMMARY OF THE INVENTION

In light of the foregoing, the invention thus provides a method of administering a compound to a patient in need thereof, comprising: subcutaneously or rectally administering a compound of Formula I in an amount sufficient to cause a pharmacological effect in said patient; wherein Formula I is as follows:

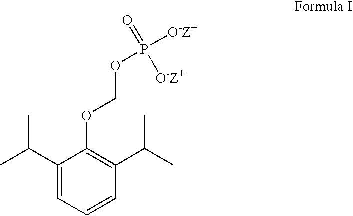

wherein each Z is independently selected from the group consisting of hydrogen, alkali metal ion, and amine. The compound is capable of causing a pharmacological effect in a patient when administered intravenously, and a substantially similar pharmacological effect when administered subcutaneously or rectally in a higher dose. Thus, in this method of the invention, the subcutaneously or rectally administered amount of the compound of Formula I is higher than the amount that would be sufficient to cause a substantially similar pharmacological effect by intravenous administration.

In a preferred aspect of this method of the invention, each Z in said compound of Formula I is an alkali metal ion. For subcutaneous administration, the compound is preferably administered in an aqueous formulation, optionally in the form disclosed in international patent application publication WO 03/057153 to Rogers et al. Alternatively, the compound of Formula I is administered rectally, and is formulated in a solid or semisolid pharmaceutical formulation, for example a suppository. Optionally, the solid or semisolid pharmaceutical formulation is adapted to release a sufficient amount of the compound into the rectal cavity either immediately, or gradually over time. Alternative formulations for rectal administration include liquid, viscous, or semisolid formulations comprising the compound of formula I in an aqueous dissolved form, or in a slurry or suspension comprising granules or particles which in turn comprise the compound of Formula I. These formulations can be further adapted to allow for specific desired release characteristics of the effective amount of the compound from the formulation into the lower digestive tract, such as fast release or sustained release over time.

The above described methods of administering the compound of Formula I to a patient, and any of the alternative or preferred embodiments thereof, include the administration of a dose sufficient to achieve a pharmacological effect in a patient. A range of doses can be selected depending largely on the pharmacological effect to be achieved. Preferred doses include those sufficient to induce or maintain an unconscious state; to induce or maintain a conscious sedated state; to induce or maintain a somnolent state, to treat insomnia, to treat sleep disorders characterized by inappropriate wakefulness; to treat anxiety; to treat nausea or vomiting; to treat itching associated with a pruritic condition; to treat an epileptic condition; to treat migraine pain; to treat cluster headaches, to treat other acute headaches, to treat trigeminal facial pain, to treat dental pain, to treat neuropathic pain, to treat phantom limb pain; to treat postoperative pain; to treat inflammatory pain; to treat neurogenic pain; and to treat arthritic pain.

Thus, one of the new and useful findings of the inventors being that the compounds of Formula I can be administered subcutaneously or rectally, the methods of this invention include, per se, methods of administering the compound of Formula I employing a range of defined doses, without being limited to the specific purpose for which they are administered. Persons of ordinary skill in the art can determine, without undue experimentation, at which dose a compound of Formula I causes a pharmacological effect (including the specific pharmacological effects recited above), and thus select appropriate doses for use in the methods of this invention. Since the methods of this invention require that subcutaneous or rectal doses be higher than therapeutically equivalent intravenous doses, one skilled in the art can determine the intravenous dose sufficient to cause a pharmacological effect, and then administer a higher dose of the compound via the therapeutically convenient subcutaneous or rectal routes to cause a substantially similar pharmacological effect. These steps require no more than routine experimentation by those skilled in the art, especially in light of the guidance and exemplary doses provided herein, and can all be done within the bounds of the invention.

In addition, the methods of this invention include methods for inducing or maintaining general anesthesia, for inducing or maintaining a conscious sedated state, and for treating a range of medical disorders such as the ones enumerated above. In a method for treating or preventing pain, a sufficient amount of the compound of Formula I is subcutaneously or rectally administered to a patient in need thereof. Preferred embodiments of this aspect of the invention include methods of treating or preventing migraine pain, cluster headaches, other acute headaches, trigeminal facial pain, dental pain, neuropathic pain, phantom limb pain; postoperative pain, inflammatory pain, neurogenic pain, and arthritic pain. In these methods for treating pain syndromes, the compound of Formula I is administered subcutaneously or rectally in an amount that is higher than the therapeutically equivalent intravenous amount.

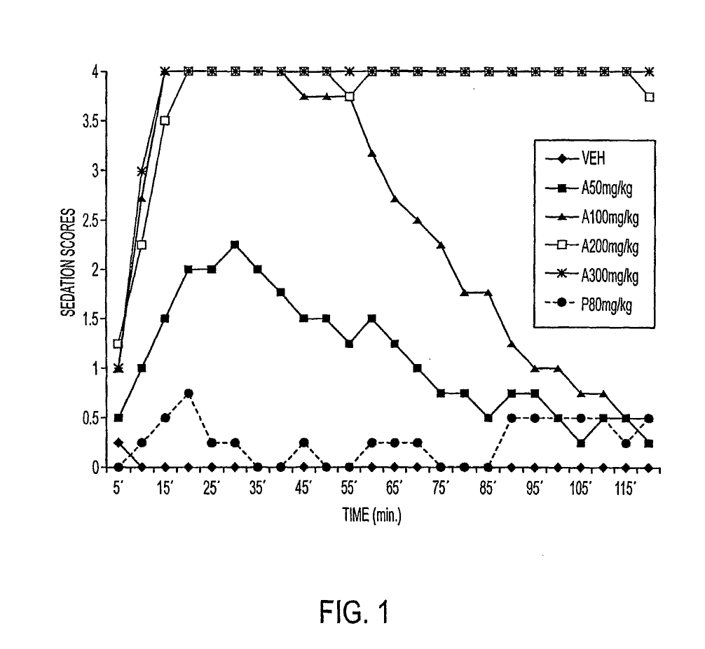

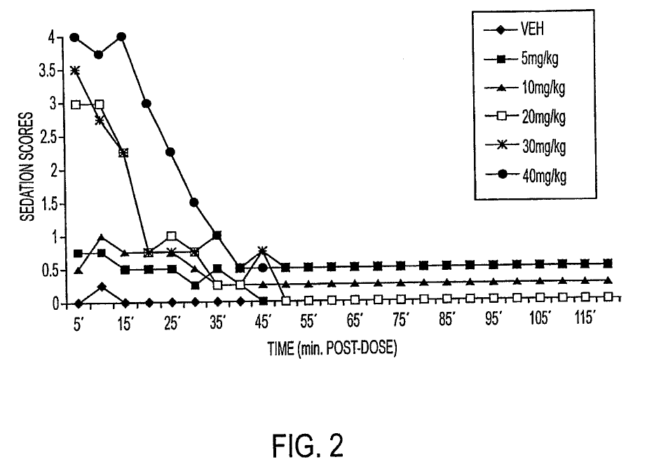

FIG. 1 illustrates the sedative/anesthetic effects of various doses of a compound of Formula I, O-phosphonooxymethylpropofol disodium salt, formulated as an aqueous solution, on rats following subcutaneous injection. Administration of the experimental compound caused a rapid onset (within 5-10 minutes of subcutaneous injection) of sedated behavior, the extent and duration of which depended on the administered dose. In contrast, subcutaneous administration of 80 mg/kg propofol in its commercially available liquid emulsion formulation (P80 mg/kg) caused only very mild or no sedative effects. For experimental details, see Example 1, below. FIG. 2 illustrates the sedative/anesthetic effects of various doses of the experimental compound when administered via the intravenous route. See Example 1, below, for experimental details.

According to one embodiment of the present invention, an unconscious state is induced or maintained in a patient by the subcutaneous or rectal administration of a prodrug of propofol in an amount sufficient to cause and maintain loss of consciousness. The prodrug is a compound of Formula I:

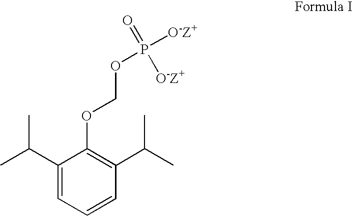

or a pharmaceutically acceptable salt thereof, wherein each Z is independently selected from the group consisting of hydrogen, alkali metal ion, and amine. Each Z preferably is an alkali metal ion, especially a sodium ion. In this embodiment of the invention, the unconscious state is induced or maintained through administration of the compound of Formula I in an amount that is higher than the amount necessary to achieve a therapeutically equivalent unconscious state through intravenous administration of the compound.

The compound of Formula I may be administered by itself or may be co-administered together with one or more additional active agents. Non-limiting examples of additional active agents include, without limitation, hypnotic, analgesic, anti-inflammatory, amnesic, muscle relaxant, and sedative agents. Such additional active agents may be incorporated into a pharmaceutical composition containing the compound of Formula I, or may be administered in a separate pharmaceutical formulation by any suitable route.

Appropriate exemplary doses for inducing or maintaining an unconscious state in a patient by single or repeated rectal administration of the compound of Formula I range from about 100 mg/kg to about 1,000 mg/kg, preferably from about 200 mg/kg to about 800 mg/kg, and more preferably from about 375 mg/kg to about 750 mg/kg.

If the unconscious state is induced or maintained by administering the compound of Formula I via the subcutaneous route, suitable exemplary doses range from about 20 mg/kg to about 500 mg/kg, preferably from more than 30 mg/kg to about 300 mg/kg.

As will be appreciated by those skilled in the art, many factors influence the choice of appropriate dosage, mode, and schedule of administration. For example, the appropriate dosage for inducing or maintaining an unconscious state in a patient, or for practicing any of the other methods of this invention recited below, may depend on whether the patient is a human, or another mammal, or is a non-mammalian patient; it may depend on the patient's age, weight, sex, diet, health, underlying medical condition, and the like. Therefore, an anesthesiologist, veterinarian, or other medical, science, or health practitioner skilled in the art will be able to devise, in light of the guidance provided herein, and without undue experimentation, an appropriate treatment protocol for practicing the present invention.

In another embodiment of the invention, a conscious sedated state is induced, or maintained over an extended period of time, in a patient by subcutaneous or rectal administration of a compound of Formula I.

In another embodiment of the present invention, a somnolent state is induced, or maintained over an extended period of time, in a patient. As is the case for a conscious sedated state, above, the somnolent state can be induced or maintained by subcutaneously or rectally administering an effective amount of a compound of Formula I.

Appropriate exemplary dose levels for inducing or maintaining a somnolent state in a patient by single or repeated bolus injection rectal administration range from about 10 mg/kg to about 400 mg/kg, preferably from about 20 mg/kg to about 300 mg/kg, and more preferably from about 25 mg/kg to about 250 mg/kg. Dose levels sufficient to induce a conscious sedated state overlap with doses sufficient to induce a somnolent state, and range from about 15 mg/kg to about 500 mg/kg, preferably from about 20 mg/kg to about 500 mg/kg, and more preferably from about 30 mg/kg to about 400 mg/kg.

If the somnolent state is induced or maintained by single or repeated subcutaneous administration the compound of Formula I, suitable exemplary doses range from about 2 mg/kg to about 250 mg/kg, preferably about 5 mg/kg to about 200 mg/kg, and more preferably about 10 mg/kg to about 150 mg/kg. Dose levels sufficient to induce or maintain a conscious sedated state overlap with those required to induce or maintain a somnolent state, and range for example from about 7 mg/kg to about 300 mg/kg, preferably from about 15 mg/kg to about 250 mg/kg, and more preferably from more than 20 mg/kg to about 200 mg/kg.

The induction or maintenance of a somnolent state, experienced as e.g. a relaxed and mildly drowsy inclination to sleep, are desirable, for example, in individuals suffering from insomnia or another condition characterized by increased and inappropriate wakefulness relative to the demands of society, such as, circadian rhythm sleep disorders (e.g. delayed sleep phase disorder, “jet lag”, or “shift work” type sleep disorder). Optionally, subcutaneous or rectal doses of the compound of Formula I can be adjusted to treat specific aspects of the sleep disorder, such as sleep latency, depth of sleep, or duration of sleep. For therapeutic use, the compound of Formula I can be administered singly, or in combination with other agents useful in the therapy of sleep disorders, combined in a single formulation or separately.

Dose levels sufficient to induce a conscious sedated state or a somnolent state are further useful in the treatment of anxiety in patients in need of such treatment, as will be appreciated by those skilled in the art. Thus, anxiolytically effective doses of the compound of Formula I will be coextensive with doses which themselves cause conscious sedation or mild to moderate sleepiness, and can be administered to patients in need of anxiolytic therapy via the therapeutically convenient subcutaneous or rectal routes.

Those skilled in the art will appreciate that compounds of Formula I, while being useful in the induction and maintenance of anesthesia, sedation, sleep, and anxiolysis as described above, are also useful in treating other medical conditions known to be amenable to treatment with propofol. Therefore, there is provided in another aspect of this invention a method of suppressing nausea or vomiting in a patient, wherein a compound of Formula I is rectally or subcutaneously administered to a patient in an amount sufficient to suppress nausea or vomiting. As in the induction and maintenance of anesthesia, sedation, sleep, and anxiolysis, the subcutaneously or rectally administered dose in this method of the invention is higher than the dose sufficient to achieve a therapeutically equivalent antiemetic effect via intravenous injection. While this method is useful in suppressing nausea and vomiting in a variety of situations, such as, for example where the patient suffers from motion sickness, it has particular applications in settings where the patient suffers from, or is at risk of, nausea or vomiting related to cancer chemotherapy or radiation therapy, or where the patient suffers from postoperative nausea and vomiting. Within this aspect of the invention, compounds of Formula I are preferably administered at subhypnotic doses, i.e. the dose of the compound of Formula I, whether administered subcutaneously or rectally, does not cause loss of consciousness, and, if the patient is not also in need of sedation; preferably does not cause a sedated state. For example, appropriate doses for suppressing or alleviating nausea and vomiting in a patient by single or repeated rectal administration range from about 0.5 mg/kg to about 450 mg/kg, preferably from about 1 mg/kg to about 400 mg/kg, and more preferably from about 5 mg/kg to about 350 mg/kg. Other effective doses may be used if the compound is administered subcutaneously. Such exemplary antiemetic doses range from about 1 mg/kg to about 180 mg/kg, preferably from about 2 mg/kg to about 150 mg/kg, and more preferably from more than 15 mg/kg to about 100 mg/kg.

Another aspect of the present invention provides a method of treating itching associated with a pruritic condition in a patient, wherein a compound of Formula I is rectally or subcutaneously administered to a patient in an amount sufficient to prevent, alleviate, or suppress localized or general itching. The amount administered in this method of the invention is higher than the amount that is sufficient to cause a therapeutically equivalent antipruritic effect by intravenous injection of the compound of Formula I. Within this aspect of the invention, compounds of Formula I are preferably administered at subhypnotic doses, i.e., the administered amount of the compound of Formula I does not cause loss of consciousness, and, if the patient is not also in need of sedation, preferably does not cause a sedated state. For example, appropriate doses for suppressing or alleviating local or generalized itching in a patient by single or repeated rectal administration range from about 0.5 mg/kg to about 450 mg/kg, preferably from about 1 mg/kg to about 400 mg/kg, and more preferably from about 5 mg/kg to about 350 mg/kg. Other effective doses may be used if the compound is administered subcutaneously. Such exemplary antipruritic doses range from about 1 mg/kg to about 180 mg/kg, preferably from about 2 mg/kg to about 150 mg/kg, and more preferably from more than 15 mg/kg to about 100 mg/kg.

The compound of Formula I, or a pharmaceutically acceptable salt thereof, may be administered for treating patients suffering from an epileptic condition. A patient in need of such treatment is rectally or subcutaneously administered a dose of a compound of Formula I in an amount sufficient to prevent, suppress, or alleviate the epileptic condition. The dose administered to practice this method of the invention is higher than the dose sufficient to cause a therapeutically equivalent anticonvulsive effect by intravenous administration of the compound of Formula I. Suitable exemplary dosages, for treating patients suffering from an epileptic condition range from subhypnotic doses, such as the antiemetic or antipruritic doses, as defined above, to higher, hypnotic doses, as required by the individual patient's needs. Individual suitable doses can be determined by those skilled in the art, especially in light of the guidance provided herein. A suitable dose for an unconscious patient presenting with status epilepticus, for example, may be determined and adjusted as needed by monitoring brain seizure activity on an electroencephalogram, and a suitable formulation comprising the compound of Formula I may be administered in the form of a suppository or via subcutaneous injection.

If an epileptic condition is to be treated by single or repeated rectal administrations of a compound of Formula I, for example, appropriate doses typically range from about 0.5 mg/kg to 1000 mg/kg, more usually from about 2 mg/kg to about 500 mg/kg, and even more usually from about 5 mg/kg to about 400 mg/kg body weight. If the epileptic condition is to be treated by subcutaneous administration of the compound of Formula I, suitable exemplary doses range from about 0.5 mg/kg to about 400 mg/kg, preferably from about 1 mg/kg to about 300 mg/kg, and more preferably from about 5 mg/kg to about 200 mg/kg body weight.

In another aspect, the present invention provides a method for treating migraine pain, cluster headaches, and other acute headaches. Patients in need of such treatment are rectally or subcutaneously administered an effective amount of a compound of Formula I, or of a pharmaceutically acceptable salt thereof, singly, or in repeated doses until pain relief is accomplished. The dose administered to practice this method of the invention is higher than the dose sufficient to cause a therapeutically equivalent analgesic effect by intravenous administration of the compound of Formula I. Exemplary subcutaneous doses suitable to practice this aspect of the invention range from about from about 2 mg/kg to about 300 mg/kg, preferably from about 5 mg/kg to about 250 mg/kg, and more preferably from about 10 mg/kg to about 200 mg/kg. When the compound is administered via the rectal route, for example in the form of a suppository, exemplary suitable doses range from about 5 mg/kg to about 500 mg/kg, preferably from about 10 mg/kg to about 500 mg/kg, and more preferably from about 20 mg/kg to about 400 mg/kg. Since such doses overlap with antiemetic doses, above, they are also expected to be effective in treating nausea frequently associated with migraine pain.

As will be appreciated by those skilled in the art, pain syndromes other than acute headaches will also be treatable by rectal or subcutaneous administration of the compounds of Formula I at the preferred dose levels described in the preceding paragraph, and the treatment of such other pain syndromes is intended to be within the scope of this invention. Non-limiting examples of such other pain syndromes are: trigeminal facial or dental pain; neuropathic pain associated with neuropathies caused by disease (e.g. diabetes, or viral infections such as herpes or HIV) or drugs (e.g. taxol, cisplatin, and other anticancer agents); phantom limb pain suffered by amputees; persistent and largely intractable postoperative pain; and arthritic pain.

The present invention also provides a method for the treatment of a pathologic condition having an inflammatory component in a patient, wherein a pharmacologically effective amount of a compound of Formula I is rectally or subcutaneously administered to the patient. This embodiment of the invention finds particular application in the treatment of a pathologic condition of the nervous system having an inflammatory component.

In another aspect, the present invention provides a method for the treatment of a pathologic respiratory condition in a patient, wherein a pharmacologically effective amount of a compound of Formula I as defined above is subcutaneously or rectally administered to the patient. This embodiment of the invention finds particular application in pathologic respiratory conditions associated with oxidative tissue damage.

In another aspect, the present invention provides a method of treatment wherein a compound of Formula I as defined above is rectally or subcutaneously administered to a patient in conjunction with a cytostatic chemotherapeutic agent, and wherein the patient suffers from cancer.

In another aspect, the present invention provides a method of treating spasticity, hyperekplexia, or of providing muscle relaxation in a patient in need thereof, which comprises rectally or subcutaneously administering to said patient a therapeutically effective amount of a compound of Formula I.

In yet another aspect of the present invention, there is provided a method of preventing neurodegeneration in the central nervous system, which comprises: rectally or subcutaneously administering to a patient suffering from, or being at risk for, neurodegeneration caused by traumatic or vascular injury, toxicity, or disease, a therapeutically effective amount of a compound of Formula I. In a preferred embodiment of this aspect of the invention, the patient suffers from, or is at risk of, ischemic injury to the brain, for example as a result of having suffered a stroke.

Methods for the chemical synthesis of the propofol prodrug of Formula I from propofol are described in U.S. Pat. No. 6,204,257 to Stella et al., and are incorporated herein by reference in their entirety. Propofol itself, as a source material for the chemical synthesis of the prodrug of the invention, is readily available to the skilled chemist from commercial suppliers. A preferred process for the chemical synthesis of the prodrug is disclosed in international patent application publication WO 03/059255 to Bonneville et al., the teachings of which are incorporated herein by reference in their entirety. The propofol prodrug of Formula I is water soluble and can be formulated in aqueous solutions or in other suitable pharmaceutical compositions suitable for subcutaneous or rectal administration.

As skilled persons will appreciate, the compounds of Formula I can be formulated for rectal or subcutaneous administration according to methods which are well-established in the art and require no more than routine experimentation. The skilled person is directed to widely available reference works, such as Gennaro's treatise “Remington: The Science and Practice of Pharmacy” (Lippincott, Williams and Wilkins (Pub.), 2003), or Ansel, Allen, and Popovich's treatise “Pharmaceutical Dosage Forms and Drug Delivery Systems” (Lippincott, Williams and Wilkins (Pub.), 2004), the teachings of which are herein incorporated by reference.

### EXAMPLE 1

This example compares the dose-dependent pharmacological effects of a propofol prodrug of Formula I, O-phosphonooxymethyl propofol disodium salt, on rats when administered in single subcutaneous injections to the pharmacological effects observed after intravenous infusion. Young adult male Sprague-Dawley rats (250-300 g, Charles River Laboratories) received subcutaneous doses of vehicle (0.12% Tris/0.25% monothioglycerol/saline; n=4) or of O-phosphonooxymethyl propofol disodium salt at doses of 50, 100, 200 and 300 mg/kg, dissolved in vehicle to achieve a uniform injection volume of 2 ml/kg body weight (n=2 per dose). The animals' behavior was then scored independently by two blinded but experienced observers in 5-minute intervals for 2 hours according to the following rating scale: 4=loss of consciousness; 3=moderate to deep sedation, markedly reduced responsiveness to external stimuli and slow but generally maintained postural reflexes; 2=“drowsy,” some slowing and sluggishness of postural reflexes but maintained responsiveness to external stimuli; 1=awake but passive, little to no locomotor or exploratory activity; 0=normal. For comparison, an additional two animals received a subcutaneous injection of propofol (80 mg/kg) in its commercially available liquid emulsion formulation. For further comparison, the above-described pharmacological effects of subcutaneous administration of the tested prodrug were compared with those caused by an intravenous infusion: Under halothane anesthesia, young adult rats received femoral vein catheters which were exteriorized and attached to a liquid swivel via a protectant spring. About 20 minutes after catheterization, and after full behavioral recovery from halothane anesthesia, each animal was attached to an electronic infusion pump and was administered vehicle, or 5, 10, 20, 30, or 40 mg/kg of the test prodrug (n=2 per dose) in 1 ml total volume by gradual constant-rate intravenous infusion over 10 minutes. Behavioral rating as described above began immediately following the end of infusion. The results of this experiment are illustrated in FIG. 2.

The results of these experiment are presented in FIGS. 1 and 2. Animals subcutaneously dosed with O-phosphonooxymethyl propofol disodium salt displayed a rapid (within 5-10 minutes of dosing) dose-dependent onset of sedated behavior, quickly followed by loss of consciousness in the 100, 200, and 300 mg/kg dose groups (see FIG. 1). Loss of consciousness lasted for more than two hours in animals in the two highest dose groups. Compared to vehicle-dosed control animals, animals in the 50 mg/kg dose group displayed signs of mild to moderate sedation lasting for about 1-1.5 hours following subcutaneous administration (see FIG. 1). In contrast, animals dosed with propofol displayed none or only very mild sedation (see FIG. 1). When the test compound was administered via intravenous infusion, the onset of sedated behavior was seen even more rapidly than after subcutaneous-injection; however the observed effects were of shorter duration compared to subcutaneous administration, and lower doses were required to achieve sedation and loss of consciousness (see FIG. 2). This study demonstrates that the tested prodrug of Formula I, O-phosphonooxymethyl propofol disodium salt, is bioavailable when dosed subcutaneously, and is capable of causing a relatively long-lasting dose-dependent anesthetic or sedative effect with a rapid onset after administration.

Upon administration, the test prodrug is converted in the body into propofol, its pharmacologically active metabolite. The pharmacokinetic profile, i.e. the blood plasma concentration of propofol derived from the test prodrug, was assessed in a separate experiment. Three male Beagle dogs (8.7-10.9 kg) received intravenous, subcutaneous, or rectal doses of the prodrug (see Tab. 1, below). Blood samples were taken at pre-dose, 2 (only for intravenous and subcutaneous), 5, 15, 30 minutes, 1, 1.5, 2, 3, 4, 6, and 8 hrs after administration of the test prodrug. Blood samples were centrifuged to obtain plasma and stored frozen until analysis. The outcome of this experiment is depicted in the following Table I:

TABLE 1
Bioavailability of Propofol from O-Phosphonooxymethyl
Propofol Disodium Salt for Various Methods of Administration

| | Dose | | AUC_{Last} | Cₘₐₓ | Tₘₐₓ |
|---|---|---|---|---|---|
| Route | (mg/kg) | Dog # | (hr × ng/ml) | (μg/mL) | (hr) |
| i.v. | 16 | 46 | 1776 | 2.57 | 0.03 |
| | | 47 | 1956 | 4.93 | 0.03 |
| | | 48 | 1451 | 4.01 | 0.03 |
| | | average | 1728 ± 256.0 | 3.84 ± 1.19 | 0.03 |
| Rectal | 16.8 | 46⁽¹⁾ | 471.4 | 0.22 | 1.0 |
| | 15.7 | 47 | 204.5 | 0.17 | 0.25 |
| | 18.4 | 48 | 46.7 | 0.09 | 0.25 |
| | | average | 240.9 ± 214.7 | 0.16 ± 0.06 | 0.50 ± 0.43 |
| subcut. | 16 | 46 | 1781 | 0.46 | 1.5 |
| | | 47 | 1484 | 0.42 | 1.0 |
| | | 48 | 701.6 | 0.29 | 0.5 |
| | | average | 1322 ± 557.7 | 0.39 ± 0.09 | 1.0 ± 0.5 |

⁽¹⁾dog 46 expelled the initial suppository dose after approximately 2 minutes. A second suppository was then administered. Nominal dosing concentrations were used in calculations
“Cmax” is the mean maximum plasma concentration;
“Tmax” is the observed time from administration of the prodrug until maximal plasma concentrations of propofol from the prodrug are reached;
“AUC_{Last}” is the area under the curve from time 0 to the last measured time point (the earlier of 8 hours or the timepoint when plasma propofol levels fell below the assay detection limit).

As is apparent from Table 1, the Cmax bioavailability of propofol derived from the subcutaneously or rectally administered test compound is in a therapeutically appreciable range, albeit it is lower relative to intravenous administration, at the tested dose level. However, when the area under the curve is measured (i.e. plasma concentrations over time), rectal and especially subcutaneous bioavailability or propofol released from the prodrug compares favorably to intravenous bioavailability, especially for subcutaneous administration, where it is on average above about 70% of that of an intravenous dose. This is consistent with the observation that subcutaneous and rectal administration of the prodrug cause plasma propofol concentrations which are sustained at higher levels over time, but do not reach the same peak concentrations as those observed after intravenous administration.

These experiments demonstrate that the experimental compound is capable of causing a sedated/anesthetized state, the onset of which is about equally rapid with subcutaneous or intravenous administration, although peak effects may be delayed with subcutaneous administration. The observed pharmacological effects are dose-dependent with both routes of administration. For rectal administration, the results of the bioavailability study displayed in Table 1 allow the reasonable extrapolation that rectal administration of the prodrug, at appropriate dose levels, is equally capable of causing a pharmacological effect in experimental subjects. Based on these experiments, it is concluded that the experimental compound is bioavailable and biologically active when given by each route of administration. In the case of subcutaneous or rectal administration, the observed bioavailability and pharmacological effects indicate that the prodrug will need to be dosed at levels which exceed those required for intravenous administration. The therapeutic convenience of being able to dose the prodrug subcutaneously or rectally is expected to outweigh the need for higher dose levels in appropriate therapeutic settings.

The invention being thus described and illustrated, it will be understood by those skilled in the art that the particular examples and embodiments can be modified in many ways without significantly departing from the scope and substance of this invention. The present application contemplates any and all such modifications.

We claim:

1. A method for inducing or maintaining an unconscious state in a patient in need thereof, comprising: subcutaneously or rectally administering to said patient a compound of Formula I, or a pharmaceutically acceptable salt thereof, in an amount sufficient to cause or maintain loss of consciousness; Formula I being: 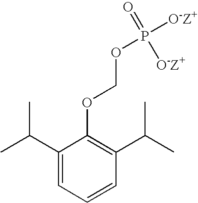
wherein each Z is independently selected from the group consisting of hydrogen, alkali metal ion, and amine; wherein said subcutaneously or rectally administered amount is higher than the amount necessary to achieve a therapeutically equivalent unconscious state through intravenous administration of the compound.

2. The method of claim 1, wherein said compound is administered rectally.

3. The method of claim 2, wherein said compound is administered rectally at a dose of about 100 mg/kg to about 1,000 mg/kg.

4. The method of claim 3, wherein said dose is from about 200 mg/kg to about 800 mg/kg.

5. The method of claim 4, wherein said dose is from about 375 mg/kg to about 750 mg/kg.

6. The method of claim 1, wherein the compound is administered subcutaneously.

7. The method of claim 6, wherein the compound is administered subcutaneously at a dose ranging from about 20 mg/kg to about 500 mg/kg.

8. The method of claim 7, wherein said dose ranges from more than 30 mg/kg to about 300 mg/kg.

9. A method for inducing or maintaining a conscious sedated state in a patient in need thereof, comprising: subcutaneously or rectally administering to said patient a compound of Formula I, or a pharmaceutically acceptable salt thereof, in an amount sufficient to cause or maintain conscious sedation; Formula I being: 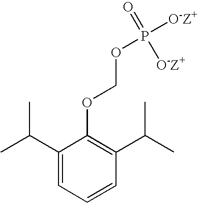
wherein each Z is independently selected from the group consisting of hydrogen, alkali metal ion, and amine.

10. The method of claim 9, wherein said subcutaneously or rectally administered amount is higher than the amount necessary to achieve a therapeutically equivalent sedated state through intravenous administration of the compound.

11. The method of claim 9, wherein the compound is administered rectally in an amount of about 15 mg/kg to about 500 mg/kg.

12. The method of claim 11, wherein the compound is administered in an amount of about 20 mg/kg to about 500 mg/kg.

13. The method of claim 12, wherein the compound is administered in an amount of about 30 mg/kg to about 400 mg/kg.

14. The method of claim 9, wherein the compound is administered subcutaneously in an amount of about 7 mg/kg to about 300 mg/kg.

15. The method of claim 14, wherein the compound is administered in an amount of about 15 mg/kg to about 250 mg/kg.

16. The method of claim 15, wherein the compound is administered in an amount of more than 20 mg/kg to about 200 mg/kg.

17. A method for inducing or maintaining a somnolent state in a subject, or for treating anxiety in a subject, comprising: subcutaneously or rectally administering to said subject a compound of Formula I, or a pharmaceutically acceptable salt thereof, in an amount sufficient to cause or maintain somnolence or anxiolysis; Formula I being: 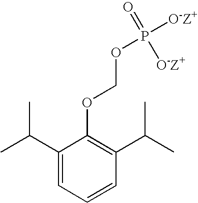
wherein each Z is independently selected from the group consisting of hydrogen, alkali metal ion, and amine.

18. The method of claim 17, wherein said subcutaneously or rectally administered amount is lower than the amount necessary to achieve a therapeutically equivalent somnolent or anxiolytic state through intravenous administration of the compound.

19. The method of claim 17 wherein the compound is administered rectally in an amount of about 10 mg/kg to about 400 mg/kg.

20. The method of claim 19, wherein the compound is administered in an amount of about 20 mg/kg to about 300 mg/kg.

21. The method of claim 20, wherein the compound is administered in an amount of about 25 mg/kg to about 250 mg/kg.

22. The method of claim 17, wherein the compound is administered subcutaneously in an amount of about 2 mg/kg to about 250 mg/kg.

23. The method of claim 22, wherein the compound is administered in an amount of about 5 mg/kg to about 200 mg/kg.

24. The method of claim 23, wherein the compound is administered in an amount of about 10 mg/kg to about 150 mg/kg.

25. A method for treating nausea or vomiting in a patient, comprising: rectally or subcutaneously administering to a patient in need thereof a Formula I, or a pharmaceutically acceptable salt thereof, in an amount sufficient to suppress nausea or vomiting; Formula I being: 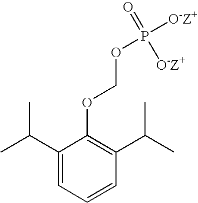
wherein each Z is independently selected from the group consisting of hydrogen, alkali metal ion, and amine.

26. The method of claim 25, wherein said subcutaneously or rectally administered amount is higher than the amount necessary to achieve a therapeutically equivalent antiemetic effect through intravenous administration of the compound.

27. The method of claim 25, wherein the compound is administered rectally in an amount of about 0.5 mg/kg to about 450 mg/kg

28. The method of claim 27, wherein the compound is administered rectally in an amount of about 1 mg/kg to about 400 mg/kg.

29. The method of claim 28, wherein the compound is administered rectally in an amount of about 5 mg/kg to about 350 mg/kg.

30. The method of claim 25, wherein the compound is administered subcutaneously in an amount of about 1 mg/kg to about 180 mg/kg.

31. The method of claim 30, wherein the compound is administered subcutaneously in an amount of about 2 mg/kg to about 150 mg/kg.

32. The method of claim 31, wherein the compound is administered subcutaneously in an amount of more than 15 mg/kg to about 100 mg/kg.

33. A method for treating localized or generalized itching, comprising:
subcutaneously or rectally administering to a patient in need thereof a Formula I, or a pharmaceutically acceptable salt thereof, in an amount sufficient to induce or maintain an antipruritic effect; Formula I being: 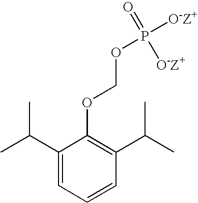
wherein each Z is independently selected from the group consisting of hydrogen, alkali metal ion, and amine.

34. The method of claim 33, wherein said subcutaneously or rectally administered amount is higher than the amount necessary to achieve a therapeutically equivalent antipruritic effect through intravenous administration of the compound.

35. The method of claim 33, wherein the compound is administered rectally in an amount of about 0.5 mg/kg to about 450 mg/kg.

36. The method of claim 35, wherein the compound is administered rectally in an amount of about 1 mg/kg to about 400 mg/kg.

37. The method of claim 36, wherein the compound is administered rectally in an amount of about 5 mg/kg to about 350 mg/kg.

38. The method of claim 33, wherein the compound is administered subcutaneously in an amount of about 1 mg/kg to about 180 mg/kg.

39. The method of claim 38, wherein the compound is administered subcutaneously in an amount of about 2 mg/kg to about 150 mg/kg.

40. The method of claim 39, wherein the compound is administered subcutaneously in an amount of more than 15 mg/kg to about 100 mg/kg.

41. A method for treating an epileptic condition in a patient, comprising: rectally or subcutaneously administering a compound Formula I, or a pharmaceutically acceptable salt thereof, in an amount sufficient to achieve or maintain an antiepileptic effect; Formula I being: 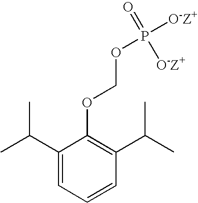
wherein each Z is independently selected from the group consisting of hydrogen, alkali metal ion, and amine.

42. The method of claim 41, wherein said subcutaneously or rectally administered amount is higher than the amount necessary to achieve a therapeutically equivalent entiepileptic effect through intravenous administration of the compound.

43. The method of claim 41, wherein the compound is administered rectally in an amount of about 0.5 mg/kg to 1000 mg/kg.

44. The method of claim 43, wherein the compound is administered rectally in an amount of about 2 mg/kg to about 500 mg/kg.

45. The method of claim 44, wherein the compound is administered rectally in an amount of about 5 mg/kg to about 400 mg/kg body weight.

46. The method of claim 41, wherein the compound is administered subcutaneously in an amount of about 0.5 mg/kg to about 400 mg/kg.

47. The method of claim 46, wherein the compound is administered subcutaneously in an amount of about 1 mg/kg to about 300 mg/kg.

48. The method of claim 47, wherein the compound is administered subcutaneously in an amount of about 5 mg/kg to about 200 mg/kg body weight.

49. A method for treating at least one pain syndrome selected from: migraine pain, cluster headaches, other acute headaches, trigeminal facial pain, dental pain, phantom limb pain, postoperative pain, arthritic pain, neuropathic pain associated with metabolic or infectious disease, and neuropathic pain associated with cancer chemotherapy; comprising: subcutaneously or rectally administering to a patient in need thereof a Formula I, or a pharmaceutically acceptable salt thereof, in an amount sufficient to induce or maintain pain relief; Formula I being: 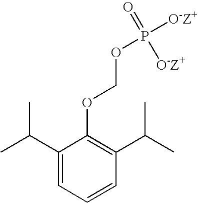
wherein each Z is independently selected from the group consisting of hydrogen, alkali metal ion, and amine.

50. The method of claim 49, wherein said subcutaneously or rectally administered amount is higher than the amount necessary to achieve a therapeutically equivalent analgesic effect through intravenous administration of the compound.

51. The method of claim 49, wherein the compound is administered subcutaneously in an amount of about 2 mg/kg to about 300 mg/kg.

52. The method of claim 51, wherein the compound is administered subcutaneously in an amount of about 5 mg/kg to about 250 mg/kg.

53. The method of claim 52, wherein the compound is administered subcutaneously in an amount of about 10 mg/kg to about 200 mg/kg.

54. The method of claim 49, wherein the compound is administered rectally in an amount of about 10 mg/kg to about 500 mg/kg.

55. The method of claim 54, wherein the compound is administered rectally in an amount of about 5 mg/kg to about 500 mg/kg.

56. The method of claim 55, wherein the compound is administered rectally in an amount of about 20 mg/kg to about 400 mg/kg.
